(19) United States
(12) Patent Application Publication
(10) Pub. No.: US 2026/0355050 A1
(43) Pub. Date: Nov. 12, 2026

(54) Regenerating Cavernous Tissue to Reverse Sexual Dysfunction

(71) Applicant: Jeffrey Nuziard, Colleyville, TX (US)

(72) Inventor: Jeffrey Nuziard, Colleyville, TX (US)

(21) Appl. No.: 19/655,500

(22) Filed: Apr. 22, 2026

Related U.S. Application Data
(60) Provisional application No. 63/794,929, filed on Apr. 25, 2025.

(51) Int. Cl. A61H 19/00; A61H 23/00; A61N 1/40

(52) U.S. Cl. CPC A61H 19/30; A61H 23/008; A61H 2201/105; A61N 1/403

(57) ABSTRACT

A treatment that regenerates cells, regenerates nerves, remodels collagen, and reverses atrophy to restore full sexual function of human genitalia.

### RELATED APPLICATION

This application claims priority to U.S. Provisional Application No. 63/794,929 filed Apr. 25, 2025, which is incorporated herein by reference in its entirety.

### TECHNICAL FIELD

The present subject matter relates to treating and reversing sexual dysfunction in male and female humans.

### BACKGROUND

Erectile dysfunction (ED) effects 7 out of 10 men at age 50, and 9 out of 10 at age 75. The first symptoms of ED usually start with the venous leak (trouble sustaining an erection, also known as venogenic ED) and progresses to being able to obtain a 75% erection and continue until complete impotence and atrophy have set in. For many years science has tried to solve erectile dysfunction with pharmaceuticals. While pharmaceuticals, such as PDE5 inhibitors, may lend a temporary effect in giving an erection, they do not address the root causes of erectile dysfunction. Many men have reported a type of resistance to these PDE5 inhibitors after 2-4 years of use.

Women also have sexual dysfunction due to degeneration of their respective genitalia.

### BRIEF DESCRIPTION OF THE DRAWINGS

The drawing figures depict one or more implementations, by way of example only, not by way of limitations. In the figures, like reference numerals refer to the same or similar elements.

Features of the various implementations disclosed will be readily understood from the following detailed description, in which reference is made to the appended drawing figures. A reference numeral is used with each element in the description and throughout the several views of the drawing. When a plurality of similar elements is present, a single reference numeral may be assigned to like elements, with an added letter referring to a specific element.

The various elements shown in the figures are not drawn to scale unless otherwise indicated. The dimensions of the various elements may be enlarged or reduced in the interest of clarity. The several figures depict one or more implementations and are presented by way of example only and should not be construed as limiting. Included in the drawing are the following figures:

FIG. 1 is a method of a REGENmax® protocol;

FIG. 2 illustrates each side of the penis shaft covering the entire corpus cavernosum treated with an Acoustic Shockwave Therapy treatment;

FIG. 3 illustrates a Bipolar radiofrequency device applied to a male penis to perform the treatment; and

FIG. 4 illustrates creating a neo-collagenesis inside the cavernous tissue using the Bipolar radiofrequency therapy.

### DETAILED DESCRIPTION

A treatment that regenerates cells, remodels collagen, and reverses atrophy to restore full sexual function of human genitalia including the male penis and the female clitoris, labia majora, and the Gräfenberg spot. The treatment includes administering hormone regenerative therapy (HRT) to the patient, administering Bipolar radiofrequency therapy to the genitalia to generate new vascularity in the genitalia, and administering a shot of a tissue regeneration composition to the genitalia to regenerate tissue.

The following detailed description includes systems, methods, techniques, instruction sequences, and computing machine program products illustrative of examples set forth in the disclosure. Numerous details and examples are included for the purpose of providing a thorough understanding of the disclosed subject matter and its relevant teachings. Those skilled in the relevant art, however, may understand how to apply the relevant teachings without such details. Aspects of the disclosed subject matter are not limited to the specific devices, systems, and method described because the relevant teachings can be applied or practice in a variety of ways. The terminology and nomenclature used herein is for the purpose of describing particular aspects only and is not intended to be limiting. In general, well-known instruction instances, protocols, structures, and techniques are not necessarily shown in detail.

The terms “coupled” or “connected” as used herein refer to any logical, optical, physical, or electrical connection, including a link or the like by which the electrical or magnetic signals produced or supplied by one system element are imparted to another coupled or connected system element. Unless described otherwise, coupled or connected elements or devices are not necessarily directly connected to one another and may be separated by intermediate components, elements, or communication media, one or more of which may modify, manipulate, or carry the electrical signals. The term “on” means directly supported by an element or indirectly supported by the element through another element that is integrated into or supported by the element.

The term “proximal” is used to describe an item or part of an item that is situated near, adjacent, or next to an object or person; or that is closer relative to other parts of the item, which may be described as “distal.” For example, the end of an item nearest an object may be referred to as the proximal end, whereas the generally opposing end may be referred to as the distal end.

The orientations of the eyewear device, other mobile devices, associated components and any other devices incorporating a camera, an inertial measurement unit, or both such as shown in any of the drawings, are given by way of example only, for illustration and discussion purposes. In operation, the eyewear device may be oriented in any other direction suitable to the particular application of the eyewear device; for example, up, down, sideways, or any other orientation. Also, to the extent used herein, any directional term, such as front, rear, inward, outward, toward, left, right, lateral, longitudinal, up, down, upper, lower, top, bottom, side, horizontal, vertical, and diagonal are used by way of example only, and are not limiting as to the direction or orientation of any camera or inertial measurement unit as constructed or as otherwise described herein.

Additional objects, advantages and novel features of the examples will be set forth in part in the following description, and in part will become apparent to those skilled in the art upon examination of the following and the accompanying drawings or may be learned by production or operation of the examples. The objects and advantages of the present subject matter may be realized and attained by means of the methodologies, instrumentalities and combinations particularly pointed out in the appended claims.

Reference now is made in detail to the examples illustrated in the accompanying drawings and discussed below.

There is a direct correlation of sustained low testosterone causing a degenerative effect to the collagen in the cavernous tissue and the smooth muscle cells (SMC) and the tunica albuginea of the penis and direct nerve damage. Furthermore, atherosclerosis of the penile vessels that occurs with ageing causes a decrease in penile oxygen tension. This correlation applies to female genitalia as well.

The decrease in penile elasticity and compliance are explained by the changes in the ratio of penile collagen that occur with aging. Contrary to the common view that testosterone deficiency or replacement only effects sexual desire, it also causes penile atrophy. There is a correlation between testosterone deficiency, cavernosal fibrosis, and ED. This disclosure details a multi-modality protocol referred to by applicant as REGENmax® that restores functionality and reverses penile fibrosis and atrophy. The REGENmax® protocol also restores functionality of female genitalia.

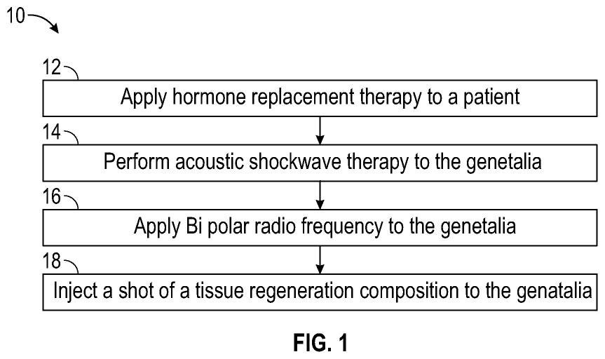

The REGENmax® protocol is a therapy as shown at 10 in FIG. 1. This protocol 10 is applicable to both the male and female genitalia. A process is first described for treating the male penis, and then for treating the female clitoris, labia majora, and the Gräfenberg spot, commonly referred to as the G-spot and which is part of the clitoral network.

At block 12, a complete hormone and blood panel is obtained on a male patient. Then, an appropriate dose of testosterone pellets is administered related to the testosterone deficiency, referred to as hormone replacement therapy (HRT). In an example, patients with lab results being below 500 total testosterone are raised to between 900 and 1100 through HRT.

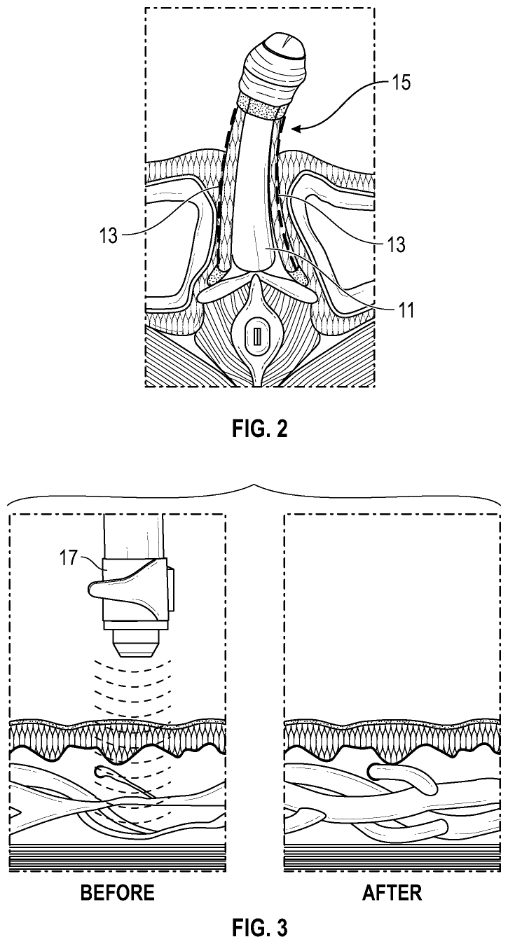

At block 14, in the same session, the trans dermis 13 on each side of the penis shaft 15 covering the entire corpus cavernosum as shown in FIG. 2 is treated with an Acoustic Shockwave treatment at the time of the blood panel. In an example, the Shockwave treatment includes a protocol using a Shockwave device 17 as shown in FIG. 3, such as a device available from Storz DA 100. In an example, power of the Shockwave device 17 has an energy setting up to 6 bars, the settings used here were between 3.0 and 3.6 bars. The trans dermis 13 on each side of the penis shaft covering the entire corpus cavernosum is pulsed 2000 times on each side of the penis, with 1000 pulses on the dorsal side, and 500 pulses on the base of the penis. This treatment aides in the creation of new vascularity, as well as micro tears in the cavernous tissue and smooth muscle cells (SMC) which yield a cellular regeneration of the tissue. The Shockwave therapy protocol creates microtraumas in the tissue stimulating a cascade of immune responses. This cascade of immune responses treats the underlying cause of erectile dysfunction by stimulating nitric oxide production leading to increased blood flow to the penis, breaks up microplaques in the small blood vessels of the penis, stimulates angiogenesis, the development of new blood vessels, and stimulating neurogenesis the formation of new nerve tissue for improved sensitivity.

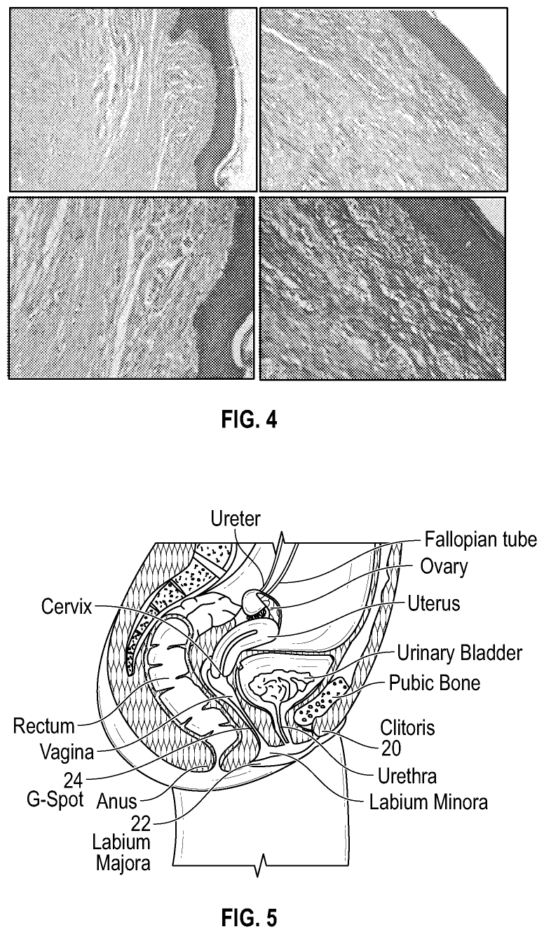

At block 16, device 17 is applied to the penis, using a Bipolar Radio Frequency device in a painting technique. The practitioner pulls the penis taught, then paints 4 minutes on each side of the external penis covering the corpus cavernosum. This process is administered at between 20 and 40 watts to to establish a temperature of the internal tissue of the penis to between 40 to 42 degrees Celsius, such as at approximately 42 °C creating a neo-collagenesis inside the cavernous tissue as illustrated in FIG. 4.

At block 18, a shot of HEshot® available from the Sexual Wellness Center of Texas, Frisco, Texas, the Applicant of this application, is injected into the penis. The HEshot® is based on a proprietary Tissue Regenerative fluid. This consists of Exosomes from the Chorion layer of the placenta, as well as type 1 and type 3 collagen, hyaluronic acid, and some highly concentrated growth factors. The application of the HEshot® to the male penis according to this disclosure is a novel treatment to treat sexual dysfunction of the male genetalia. A SHEshot®, also available from the Sexual Wellness Center of Texas, is applied to the genitalia of women. In example, the Heshot® only needs to be given one time, and that is on their second treatment. In an example, the HEshot® is administered using a 1 ml syringe with a 31 g needle. 1 ml of HEshot® is drawn and 0.5 ml is injected into the corpus cavernosum at a 45° angle into the mid shaft on each side of the penis. The HEshot® is 800 times more regenerative than Platelet-rich plasma (PRP), and it works at the molecular and cellular level to regenerate new tissue in the genitalia.

In an example, the REGENmax® protocol 10 includes treatments that continue every other week 6-8 times. At that point, a period of six weeks is given as a regeneration time. The REGENmax® protocol 10 treatments then continue for another 6-8 times every other week. Verbal evaluations are taken with a therapy practitioner at each treatment. The HRT continues every 16 weeks with new labs and continued pellet therapy.

### Study

In a prospective clinical study, 200 men aged 49-75 years with ≥12 months of erectile dysfunction (ED) underwent one year of REGENmax® therapy 10. The protocol 10 combined: (1) Bipolar Radio Frequency, (2) REGENwave® acoustic shockwave therapy, (3) bioidentical testosterone pellet hormone replacement therapy (HRT), and (4) a single intracavernosal injection of Tissue Regenerative Fluid (Chorion layer exosome compounded biologic)(TRF, HEshot®). Erectile function was graded on a 1-5 severity scale pre-and post-treatment. Statistical analysis included paired t-tests and logistic regression to assess predictors of treatment success.

Results: After 12 months, 100/200 participants who received the REGENmax® protocol, 97/100(97.0 %) achieved ≥85% functional recovery of erectile capacity with penetration-sustainable rigidity. 50/100 received Bipolar Radio Frequency as a stand-alone procedure. 27/50 achieved ≥85% functional recovery of erectile capacity with penetration-sustainable rigidity. 50/100 were treated with shockwave only, as a placebo, and 2/50(0.04%) achieved ≥85% functional recovery of erectile capacity with penetration-sustainable rigidity. The 3 non-responders of REGENmax® (3.0%) were predominantly type II diabetics (n=3, 100%). It is also noted that 21/100 were prostate cancer survivors after completing a complete prostatectomy had lost all sensitivity and sexual sensation and were unable to have an orgasm. All 21/21 had complete regeneration of nerves due to the entire protocol and are able to perform and have a complete orgasm.

Conclusion: REGENmax® therapy 10 demonstrated high efficacy in reversing ED associated with cavernosal fibrosis and testosterone deficiency. The combined regenerative approach—addressing vascular, hormonal, nerve regeneration, and collagen-remodeling pathways—offers a minimally invasive alternative for patients who are unresponsive to conventional treatments. Further randomized controlled trials are warranted to confirm durability and explore broader applications in other forms of ED.

With respect to treating females with issues including sexual dysfunction, the REGENmax® protocol 10 using the SHEshot® is administered to the female genitalia including the clitoris, the labia majora, and the G-Spot.

The REGENmax® protocol 10 is done in the same order and using the same techniques used with men. The Bipolar radiofrequency procedure is done intra-vaginally for all atrophy, incontinence, and vaginal tightness issues as well as extra-vaginally for labia regeneration. The Bipolar radiofrequency procedure is done trans clitoral, and the SHEshot® is injected 0.5ml into the clitoris and 0.5ml into the G-spot. HRT is done on women as well.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A method of treating sexual dysfunction of genitalia of a human patient, comprising:
administering hormone regenerative therapy (HRT) to the patient;
administering acoustic shockwave therapy to the genitalia;
administering Bipolar radiofrequency therapy to the genitalia to remodel collagen in the genitalia; and
administering a shot of a tissue regeneration composition to the genitalia to regenerate tissue and nerves in the genitalia.

2. The method as specified in claim 1, wherein the shot is a HEshot®.

3. The method as specified in claim 1, wherein the shot is a SHEshot®.

4. The method as specified in claim 1, wherein the Bipolar radiofrequency therapy is a administered to establish a temperature of the genitalia of between 40 and 42 degrees Celsius.

5. The method as specified in claim 1, wherein the acoustic shockwave therapy generates micro tears in cavernous tissue and smooth muscle cells (SMC) and a corpus cavernosum to yield a cellular regeneration of the tissue.

6. The method as specified in claim 5, wherein the acoustic shockwave therapy administers between 2000 and 3000 pulses per side of a penile shaft of a male human patient.

7. The method as specified in claim 1, wherein the Bipolar radiofrequency therapy creates a neo-collagenesis inside cavernous tissue of the genitalia.

8. The method as specified in claim 1, wherein a dermis of female genitalia is administered the shot.

9. The method as specified in claim 1, wherein a dermis of a clitoris is treated with the Bipolar radiofrequency therapy.

10. The method as specified in claim 1, wherein a dermis of a labia majora is treated with the Bipolar radiofrequency therapy.

11. A method of treating sexual dysfunction of a penis of a male human patient, comprising:
administering hormone regenerative therapy (HRT) to the patient;
administering acoustic shockwave therapy to the penis of the patient to generate micro tears in cavernous tissue and smooth muscle cells (SMC), creating cellular regeneration of the cavernous tissue and generating new vascularity in the penis; and
administering a shot of a tissue regeneration composition to the penis to regenerate the cavernous tissue.

12. The method as specified in claim 11, wherein the shot is a HEshot®.

13. The method as specified in claim 11, wherein a trans dermis on each side of the penis covering a corpus cavernosum is treated with the acoustic shockwave therapy.

14. A method of treating sexual dysfunction of a clitoris and a Gräfenberg spot of a female human patient, comprising:
administering hormone regenerative therapy (HRT) to the patient;
administering Bipolar radiofrequency therapy to the clitoris and the Gräfenberg spot of the patient to generate micro tears in tissue and smooth muscle cells (SMC), creating cellular regeneration of the tissue and generating new vascularity in the clitoris and the Gräfenberg spot;
administering a Bipolar radiofrequency therapy to the clitoris and the Gräfenberg spot to create neo-collagenesis inside tissue of the clitoris and the Gräfenberg spot; and
administering shot of a tissue regeneration composition to the clitoris and the Gräfenberg spot to regenerate the tissue.

15. The method as specified in claim 14, wherein the shot is a SHEshot®

16. The method as specified in claim 15, wherein a labia majora of the patient is also administered the Bipolar radiofrequency therapy and the SHEshot®.
